(19) United States
(12) Patent Application Publication
(10) Pub. No.: US 2024/0342325 A1
(43) Pub. Date: Oct. 17, 2024

(54) Ultraviolet Disinfection Systems and Methods

(71) Applicant: Bard Access Systems, Inc., Salt Lake City, UT (US)

(72) Inventors: Robin Scott Urry, Syracuse, UT (US); Dustin Payne, West Jordan, UT (US); Debra K. Cloward, Springville, UT (US); Matthew J. Prince, Herriman, UT (US); Timothy M. Snelling, Salt Lake City, UT (US); Craig Hayden, Sandy, UT (US); Taylor A. Brown, Lehi, UT (US); Jacob M. Sobelman, Salt Lake City, UT (US); Paul T. Westwood, Kaysville, UT (US); Stefan Josef Fellner, Salt Lake City, UT (US); Steffan Sowards, Salt Lake City, UT (US); Christopher Quach, North Salt Lake, UT (US); Amelia Marie Smith, Rochester, MI (US); William Robert McLaughlin, Bountiful, UT (US); Christopher Winland, Bountiful, UT (US)

(21) Appl. No.: 18/633,354

(22) Filed: Apr. 11, 2024

Related U.S. Application Data
(60) Provisional application No. 63/458,500, filed on Apr. 11, 2023.

(51) Int. Cl. A61L 2/00

(52) U.S. Cl. CPC A61L 2/0047; A61L 2202/11; A61L 2202/14; A61L 2202/16; A61L 2202/22

(57) ABSTRACT

Ultraviolet light disinfection systems expose medical devices, surfaces, patients and healthcare clinicians to UV light having wavelengths consist with killing microorganisms. UV light systems may use logic to govern the operation of UV light sources so as to apply a disinfecting dose of the UV light. UV detectors measure UV light exposure. Enclosures contain medical devices and flood the medical devices with the UV light. Some enclosures accommodate a person. Some UV systems project UV light onto high-touch surfaces of a healthcare environment. Some systems may be deployed within a patient transport vehicle. Some UV systems may be incorporated into medical devices.

### PRIORITY

This application claims the benefit of priority to U.S. Provisional Application No. 63/458,500, filed Apr. 11, 2023, which is incorporated by reference in its entirety into this application.

### BACKGROUND

Infections contracting with a healthcare environment continue to affect patient health and health care costs. The complexities around routinely disinfecting surfaces and tools can contribute to lack of following sterility protocols. Furthermore, medical devices may be damaged by heat, gas exposure and radiation of current disinfecting processes. Exposure to ultraviolet light has been shown to kill microorganisms and thus reduce infection. However, the use of UV light exposure do disinfect surfaces and medical devices may not be incorporated into sterility protocol as much as it could be.

Disclosed here are systems, devices and methods that utilize UV light to disinfect surfaces and devices in a healthcare setting.

### SUMMARY

Disclosed herein is a medical disinfection system that, according to some embodiments, includes a handheld disinfection device. The handle held disinfection device includes a device body and a number of ultraviolet (UV) light sources coupled with the device body, where the UV light source are configured to project a UV light having away from a projection surface of the device body. The handle held disinfection device further includes a console operatively coupled with the UV light source, where the console includes a processor and a memory having stored thereon logic that, when executed by the processor, causes operations of the handheld disinfection device including (i) activating the UV light source to project the UV light, and (ii) deactivating the UV light source. In some embodiments, the UV light includes a wavelength within the range of 207 nm to 222 nm.

In some embodiments, the UV light is configured to disinfect a skin surface of a patient prior to breaching a dermal layer, and in some embodiments, breaching the dermal layer includes accessing a vasculature.

In some embodiments, the operations include activating the UV light source in response to pressing a button disposed on the device body.

In some embodiments, the handheld disinfection device further includes a proximity sensor configured to determine a distance between the projection surface and the skin surface, and the operations further include preventing activation the UV light source unless the projection surface is positioned within a predefined distance from the skin surface.

In some embodiments, the operations further include preventing activation the UV light source unless the projection surface is positioned within a predefined distance from the skin surface.

In some embodiments, the operations further include deactivating the UV light source after a predefined duration of UV light activation.

In some embodiments, the handheld disinfection device is combined with at least one of an ultrasound probe, a sterile remote controller, or an adherable arm band.

In some embodiments, the handheld disinfection device includes a UV shield having a cylindrical wall extending around and away from the projection surface of the device body, where the UV shield is configured to limit UV exposure to a disinfection area of the skin surface proximate an open end of the UV shield. In some embodiments, a material of the shield includes one or more of cerium oxide, titanium oxide, carbon black, plastic acrylics, polystyrene, or polycarbonate.

In some embodiments, the UV shield is configured to contact the skin surface at the open end, and a length of the UV shield to defines a minimum distance between the skin surface and the projection surface. In some embodiments, the handheld disinfection device further includes a contact sensor coupled with the console, where the contact sensor is configured to detect a physical contact between the open end of the shield and the skin surface, and the operations further include at least one of (i) preventing activation of the UV light source in the absence of the physical contact, or (ii) activating the UV light source in response to the physical contact.

Also disclosed herein is a method of disinfecting a skin surface of a patient that, according to some embodiments, includes positioning a handheld disinfection device proximate the skin surface, where the hand held device includes a device body and a number of ultraviolet (UV) light sources coupled with the device body, and where the UV light source are configured to project a UV light away from a projection surface of the device body. The handheld disinfection device further includes a console operatively coupled with the UV light source, where the console includes a processor and a memory having stored thereon logic that, when executed by the processor, causes operations of the handheld disinfection device, and where the method further includes activating the UV light source to disinfect the skin surface.

In some embodiments of the method, activating the UV light source to disinfect the skin surface is performed in preparation of performing a vascular access procedure, and in some embodiments of the method, activating the UV light source includes pressing a button disposed on the device body.

In some embodiments of the method, the handheld disinfection device further includes a proximity sensor configured to determine a distance between the projection surface and the skin surface, and logic of the handheld disinfection device prevents activation of the UV light source unless the projection surface is positioned within a predefined distance from the skin surface. In such embodiments, the method further includes positioning the projection surface within the predefined distance to allow activation of the UV light source.

In some embodiments of the method, the logic of the handheld disinfection device automatically deactivates the UV light source after a predefined duration of UV light activation.

In some embodiments of the method, the handheld disinfection device includes a UV shield having a cylindrical wall extending around and away from the projection surface of the device body, where the UV shield is configured to limit UV exposure to a disinfection area of the skin surface proximate an open end of the UV shield. The UV shield is configured to contact the skin surface at the open end, and a length of the UV shield to defines a minimum distance between the skin surface and the projection surface.

In some embodiments of the method, the handheld disinfection device further includes a contact sensor coupled with the console, where the contact sensor is configured to detect a physical contact between the open end of the shield and the skin surface, and where activating the UV light source includes contacting the skin surface with the open end of the shield.

Also disclosed herein is a medical disinfection system that, according to some embodiments, includes a handheld disinfection device that includes (i) a device body; (ii) an ultraviolet (UV) light source coupled with the device body, where the UV light source is configured to project a UV light away from a projection surface of the device body; and (iii) a UV shield disposed proximal the UV light source, where the UV shield is configured to prevent passage of UV light therethrough and allow passage of visible light therethrough. In some embodiments, the UV light is configured to disinfect a skin surface of a patient.

In some embodiments, the UV shield is positioned between a clinician and the UV light source so that the clinician is protected from exposure to the UV light. In some embodiments, the UV shield extends laterally upward away from the device body. In some embodiments, a wall of the UV shield is formed of a refractory material such that UV light entering the wall is prevented from exiting the wall.

In some embodiments, the handheld disinfection device further includes a visible light source coupled with the device body, where the visible light source is configured to visibly illuminate the skin surface.

In some embodiments, the device further includes a console operatively coupled with the UV light source, where the console includes a processor and a memory having stored thereon logic that, when executed by the processor, causes operations, including deactivating the UV light source after a defined duration.

In some embodiments, the defined duration includes at least one of (i) any one of a number of preset time increments stored in the memory, or (ii) a time increment set by the clinician.

In some embodiments, the handheld disinfection device further includes a proximity sensor configured to determine a distance between the projection surface and the skin surface, and the operations further include preventing activation the UV light source unless the projection surface is positioned within a predefined distance from the skin surface.

Also disclosed herein is a method of disinfecting a skin surface of a patient that, according to some embodiments, includes positioning a handheld disinfection device proximate the skin surface. The hand held device includes a device body and an ultraviolet (UV) light source coupled with the device body, and the UV light source is configured to project a UV light away from a projection surface of the device body. The method further includes (i) activating the UV light source to disinfect the skin surface and (ii) viewing the skin surface through a UV light shield of the handheld disinfection device.

In some embodiments, the method further including activating a visible light source of the handheld disinfection device to visibly illuminate the skin surface.

In some embodiments of the method, the handheld disinfection device further includes a proximity sensor configured to determine a distance between the projection surface and the skin surface and a console operatively coupled with the UV light source and the proximity sensor. The console includes a processor and a memory having stored thereon logic that, when executed by the processor, causes operations of the handheld disinfection device that include preventing activation of the UV light source unless the projection surface is positioned within a predefined distance from the skin surface. In such embodiments, the method further includes positioning the projection surface within the predefined distance to allow activation of the UV light source.

In some embodiments of the method, the logic of the handheld disinfection device automatically deactivates the UV light source after a defined duration of UV light activation. In some embodiments, the defined duration includes at least one of (i) any one of a number of preset time increments stored in the memory or (ii) a time increment set by the clinician.

Also disclosed herein is a medical disinfection system that, according to some embodiments, includes an enclosure configured for placement of a person therein and a plurality of ultraviolet (UV) light sources coupled with the enclosure, where the UV light sources are configured to project UV light onto a person exterior to disinfect the person exterior.

In some embodiments, a perimeter wall of the enclosure is configured to contain the UV light within the enclosure. In some embodiments, the UV light sources are dispersed across an interior surface of the perimeter wall, the UV light sources configured to project the UV light radially inward onto the person exterior.

In some embodiments, the system further includes a frame within the enclosure, where the frame is rotatable about a centrally located vertical axis, and where the UV light sources are attached to the frame such that rotation of the frame causes the UV light sources to travel along a circular path around the person.

In some embodiments, the UV light sources are arranged to define a plane of UV light such that rotation of the frame causes projection of the UV light around an entire circumference of the person exterior.

In some embodiments, the system further includes a UV light detection system configured to detect a UV light exposure on the person exterior. In some embodiments, where the UV light detection system is configured to determine an intensity of the UV light exposure. In some embodiments, the UV light detection system includes a first UV light detector coupled with the enclosure, where the first UV light detector is configured to detect UV light emanating from the person exterior. In some embodiments, the UV light detection system includes a second UV light detector attached to the person exterior, where the second UV light detector is configured to detect the UV light received by the person exterior.

In some embodiments, the system further includes a console operatively coupled with the UV light sources and the UV light detection system, the console including a processor and a memory having stored thereon logic that, when executed by the processor, causes operations of the medical disinfection system that include one or more of (i) activating the UV light sources, (ii) recording a disinfecting event, or (iii) determining a profile of UV light exposure on the person exterior. In some embodiments, the profile of UV light exposure includes a coverage assessment of the UV light exposure.

Also disclosed herein is a method of disinfecting a person exterior that, according to some embodiments, includes (i) placing the person within an enclosure of a medical disinfection system having a plurality of ultraviolet (UV) light sources coupled with the enclosure, where the UV light sources are configured to project UV light onto a person exterior of the person to disinfect the person exterior; and (ii) activating the UV light sources.

In some embodiments of the method of disinfecting a person, the UV light sources are coupled with a rotatable frame within the enclosure, and the method further includes rotating the frame around the person.

In some embodiments of the method of disinfecting a person, the medical disinfection system further includes a UV light detection system configured to detect a UV light exposure on the person exterior, and the method further includes determining a profile of UV light exposure on the person exterior.

In some embodiments the method of disinfecting a person further includes recording a disinfecting event, where parameters of the disinfecting event include one or more of (i) a date of the disinfecting event; (ii) a time of the disinfecting event; (iii) a duration of the UV light source activation; (iv) an intensity of UV light exposure on the person exterior; or (v) the profile of UV light exposure.

Also disclosed herein is a medical disinfection system that, according to some embodiments, includes a UV light system coupled with a patient transport vehicle, where the UV light system includes a number of ultraviolet (UV) light sources configured to expose objects within the patient transport vehicle to UV light to disinfect the objects.

In some embodiments, the patient transport vehicle includes at least one of an ambulance or a helicopter. In some embodiments, windows of the patient transport vehicle are configured to inhibit transmission of the UV light therethrough.

In some embodiments the UV light sources are configured to attached to inside surfaces of the patient transport vehicle, and in some embodiments, the UV light system includes reflective surfaces configured to reflect the UV light, thereby further exposing the objects to the UV light.

In some embodiments, the UV light system is configured to activate the UV light sources during periods of non-operation of the patient transport vehicle.

In some embodiments, the UV light system is portable between at least a first compartment and a second compartment of the patient transport vehicle. In some embodiments, the first compartment includes a medical personnel compartment and UV light system is configured to disinfect objects within the medical personnel compartment. In some embodiments, the UV light system is configured to deactivate the UV light sources when an occupant is present within the medical personnel compartment. In some embodiments, the second compartment includes a patient compartment and UV light system is configured to disinfect objects within the patient compartment. In some embodiments, the UV light sources are coupled with an overhead ceiling surface of the patient compartment.

In some embodiments, the UV light system includes a remote control such that a user may remotely activate and deactivate the UV light sources, and in some embodiments, the UV light system includes a timer configured to deactivate the UV light sources after a defined activation time period.

In some embodiments, the UV light system is electrically coupled with an electrical system of the patient transport vehicle, and in some embodiments, the UV light system includes a battery power source.

Also disclosed herein is a medical disinfection system that, according to some embodiments, includes an ultraviolet (UV) light system configured to expose a high-touch surface of a patient care environment to a UV light configured to disinfect the high-touch surface, the UV light system comprising an ultraviolet (UV) light source optically coupled with the high-touch surface. In some embodiments, the high-touch includes one of a handle or a latch.

In some embodiments, the high-touch surface is an exterior surface of an object, where the object includes a material transparent to the UV light, and where the UV light source is disposed beneath the exterior surface such that the UV light propagates through the material to the exterior surface.

In some embodiments, the UV light source is configured to project the UV light onto the high-touch surface. In some embodiments, the UV light source is configured to focus the UV light onto the high-touch surface.

In some embodiments, the UV light system includes a shroud configured to at least partially encapsulate the high-touch surface, and the UV light source is coupled to an inside surface of the shroud. In some embodiments, the inside surface includes a reflective surface.

In some embodiments, the shroud is configured to transition between (i) an deployed position, wherein the shroud at least partially encapsulates the high-touch surface; and a retracted position, wherein the shroud is disposed away from the high-touch surface to allow access to the high-touch surface.

In some embodiments, the UV light source includes an array of light emitting diodes. In some embodiments, the UV light source includes an optical fiber, and in some embodiments, the optical fiber is configured to project the UV light laterally away from the optical fiber along a length of the optical fiber.

Also disclosed herein is a medical disinfection system that, according to some embodiments, includes an ultraviolet (UV) light system coupled with a continuous positive airway pressure (CPAP) system, where the UV light system includes a UV light source extending along a tube of the CPAP system, and where the UV light source is configured to expose an interior of the tube to a UV light configured to disinfect the interior of the tube.

In some embodiments, the UV light source is disposed within the tube.

In some embodiments, the tube includes a material that is not damaged by the UV light.

In some embodiments, the UV light system is battery operated.

In some embodiments, the UV light source is selectively (i) inserted within the tube and (ii) removed from the tube. In some embodiments, the UV light source includes at least one of a stylet, a probe or a scope.

In some embodiments, the UV light system includes a notification system configured to indicate a status of a disinfection cycle of the UV light system, and the notification system includes at least one of a visual indicator or an audible indicator.

In some embodiments, the UV light system is electrically coupled with the CPAP system. In some embodiments, the notification system is incorporated into the CPAP system.

In some embodiments, the UV light system is configured to automatically initiate the disinfection cycle.

In some embodiments, the UV light system includes a sensor configured to detect a contamination of the tube, and the UV light system is configured to initiate the disinfection cycle based on a detection of the contamination.

Also disclosed herein is a medical disinfection system that, according to some embodiments, includes an ultraviolet (UV) light system coupled with a saliva ejector, where the UV light system includes an elongate member configured for placement with a lumen of the saliva ejector. The elongate member includes a UV light source configured to expose an inside surface of the lumen to a UV light configured to disinfect the inside surface.

In some embodiments, the UV light source includes an optical fiber. In some embodiments, the optical fiber is configured to project the UV light radially outward from the optical fiber along a length of the optical fiber. In some embodiments, the optical fiber is includes a coating configured to disperser the UV light within the lumen.

In some embodiments, the UV light source is configured for insertion into the lumen from proximal end of the lumen. In some embodiments, the UV light source is configured for repeatable placement within and removal from the lumen so as to repeatedly disinfect the inside surface, and in some embodiments, the UV light source is configured for storage within a casing of the saliva ejector when the UV light source is removed from the lumen.

In some embodiments, the UV light system includes a liquid absorbing device coupled to the elongate member, where the liquid absorbing device is configured to remove a liquid from the lumen. In some embodiments, wherein the liquid absorbing device includes a disposable cloth member configured for attachment to and detachment from the elongate member.

Also disclosed herein is a medical disinfection system that, according to some embodiments, includes an enclosure configured for placement of medical devices therein and an ultraviolet (UV) light source coupled with the enclosure, where the UV light source is configured to expose the medical devices to a UV light to disinfect the medical devices. In some embodiments, the medical devices include dental instruments.

In some embodiments, the enclosure is a storage cabinet for the medical devices. In some embodiments, the enclosure is configured for use within a patient transport vehicle. In some embodiments, a perimeter wall of the enclosure is configured to contain the UV light within the enclosure.

In some embodiments, the UV light source includes a plurality of individual UV light sources arranged in an array within the enclosure so that an entire interior of the enclosure is exposed to the UV light. In some embodiments, the plurality of UV light sources are dispersed across an interior surface of the perimeter wall, the UV light sources configured to project the UV light inward onto the medical devices.

In some embodiments, the enclosure includes a number of shelves configured for placement of the medical devices thereon. In some embodiments, the shelves are configured for transmission of the UV therethrough. In some embodiments, one or more of the UV light sources are disposed between adjacent shelves.

In some embodiments, the system further include a number of cassettes configured for placement of the medical devices therein, where the cassettes are configured for placement within the enclosure, and where the cassettes are formed of a material transparent to the UV light.

In some embodiments, at least a first subset of the UV light sources include light emitting diodes, and in some embodiments, at least a second subset of the UV light sources include optical fibers, where each optical fiber is configured to project the UV light laterally away from the optical fiber.

In some embodiments, the system further includes a console operatively coupled with the UV light source. The console includes a processor and a memory having stored thereon logic that, when executed by the processor, causes operations that include activating the UV light source.

In some embodiments, the operations further include receiving input from a user and adjusting a duration of UV light activation based on the input from the user.

In some embodiments, the system further includes a medical device identification system configured to identify at least one medical device disposed within the enclosure. In some embodiments, the medical device identification system is coupled with the console and the operations further include adjusting the duration of UV light activation based on an identification of the at least one medical device.

In some embodiments, the system further includes a temperature control system configured to at least one of warm or cool the medical devices disposed within the enclosure.

Also disclosed herein is a medical disinfection system that, according to some embodiments, includes a plurality of UV light sources arranged in a two-dimensional array and a frame coupled with the plurality of UV light sources, where the frame defines a disinfection area. The plurality of UV light sources define a deflectable surface extending across the disinfection area, where the deflectable surface is configured to receive a medical device thereon such that the deflectable surface conforms to an external surface of the medical device, and where the plurality of UV light sources are configured to project a UV light away from the deflectable surface onto the external surface to disinfect the external surface.

In some embodiments, each UV light source includes an elongate member, and each UV light source is configured to project the UV light away from a free end of the elongate member. In some embodiments, each UV light source is directly coupled with the frame. In some embodiments, the frame includes a plate extending across the disinfection area, and each UV light source is individually displaceable with respect to the plate. In some embodiments, the plate includes a plurality of apertures extending through the plate, and each elongate member is slidably disposed within a corresponding aperture.

In some embodiments, each UV light source includes a light guide extending along the elongate member, and in some embodiments, each light guide includes an optical fiber.

Also disclosed herein is a medical device package that, according to some embodiments, includes a container configured for exposure to an ultraviolet (UV) light, the UV light configured to disinfect medical devices. The container includes first and second compartments, where each of the first and second compartments includes an inside wall surface configured to reflect the UV light. The container further includes a cover that includes a first cover portion extending across an open end of the first compartment and a second cover portion extending across an open end of the second compartment. A first medical device is disposed within the first compartment, where the first medical device is configured to receive a first dose of the UV light to disinfect the first medical device, and a second medical device is disposed within the second compartment, where the second medical device configured to receive a second dose of the UV light to disinfect the second medical device, and where the second dose greater than the first dose.

In some embodiments, the first cover portion is configured to allow the first dose of the UV light to enter the first compartment to disinfect the first medical device, and the second cover portion is configured to allow the second dose of the UV light to enter the second compartment to disinfect the second medical device.

In some embodiments, the cover includes multiple layers, and at least a subset of the multiple layers are configured to limit transmission of the UV light therethrough. In some embodiments, the first cover portion includes a first number of the multiple layers and the second cover portion includes a second number of the multiple layers, where the second number is less than the first number. In some embodiments, at least one of the first or second cover portions includes the cover having one or more of the multiple layers removed therefrom.

In some embodiments, at least one of the first or second compartment includes a UV light pathway extending between an interior and exterior of the at least one of the first or second compartments, and in some embodiments, the UV light pathway includes one or more of a tube, a window, a light pipe, or an optical fiber.

In some embodiments, at least one of the first or second compartments includes a probe at least partially disposed within the compartment, the probe configured to emit the UV light.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates an ultraviolet light disinfection system including a handheld disinfection device configured for projecting ultraviolet light onto a skin surface, in accordance with some embodiments.

FIG. 2 illustrates an ultraviolet light disinfection system including a handheld disinfection device configured for projecting ultraviolet (UV) light onto a skin surface and further including a shield to protect the user from UV light exposure, in accordance with some embodiments.

FIG. 3A illustrates an ultraviolet light disinfection system configured for disinfecting an exterior surface of a medical person, in accordance with some embodiments.

FIG. 3B illustrates another embodiment of an ultraviolet light disinfection system configured for disinfecting an exterior surface of a medical person, where the system includes a rotating frame, in accordance with some embodiments.

FIG. 4 illustrates an ultraviolet light disinfection system configured for disinfecting a patient transport environment, in accordance with some embodiments.

FIGS. 5A-5E illustrate various embodiments of an ultraviolet light disinfection system configured for disinfecting high-touch areas of a healthcare environment, in accordance with some embodiments.

FIGS. 6A-6C illustrate various embodiments of an ultraviolet light disinfection system configured for disinfecting an interior portion of a continuous positive airway pressure (CPAP) machine, in accordance with some embodiments.

FIGS. 7A-7B illustrate embodiments an ultraviolet light disinfection system including a probe configured for disinfecting an interior of a saliva ejector, in accordance with some embodiments.

FIGS. 8A-8B illustrate an ultraviolet light disinfection system configured for projecting ultraviolet light onto medical devices disposed within a container, in accordance with some embodiments.

FIGS. 9A-9B illustrate an ultraviolet light disinfection system including a plurality of discreetly adjustable light guides, in accordance with some embodiments.

FIGS. 10A-10D illustrate medical device packages including a plurality of cavities individually covered by an ultraviolet light opaque material, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, optical, magnetic, electromagnetic, fluid, and thermal interaction. Two components may be coupled with each other even though they are not in direct contact with each other. For example, two components may be coupled with each other through an intermediate component. Two components that are directly coupled with each other may be in direct contact with each other.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

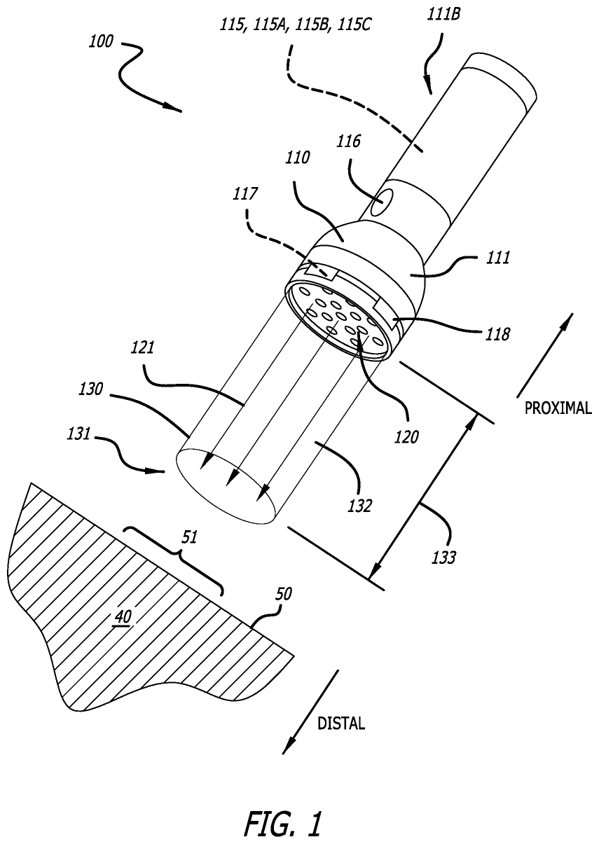

FIG. 1 illustrates an ultraviolet light disinfection system 100 including a handheld disinfection device 110 configured for projecting ultraviolet light 121, in accordance with some embodiments. The system 100 is generally configured to disinfect a skin surface 50 of a patient 40 in accordance with a medical procedure prior, such as breaching a dermal layer, for example. More specifically, the system 100 is configured to disinfect an insertion site area 51 of the skin surface 50 in preparation for accessing a vasculature of the patient 40 and in some embodiments, breaching the dermal layer includes accessing a vasculature.

The handheld disinfection device (device) 110 includes a device body 111 defining a distal end 111A and a proximal end 111B. The device 110 includes a UV light source 120 coupled with the device body 111 where UV light source 120 may include any number (e.g., 1, 2, 5, 10, 20 or more) of individual light sources (e.g., light emitting diodes). The UV light source 120 is configured to project the UV light 121 away from a projection surface 123 of the device body 111. The device 110 further includes a console 115 operatively coupled with the UV light source 120. The console 115 includes a processor 115A, a memory 115B (e.g., non-transitory computer-readable medium), and a power source 115C (e.g., a battery). Logic stored on the memory, when executed by the processor, performs operations of the device 110, where the operations may include (i) activating the UV light source 120 to project the UV light 121 and/or (ii) deactivating the UV light source to halt the projection of the UV light 121.

The UV light 121 may include any wavelength spectrum suitable for disinfecting a surface. For example, the UV light may include a UV-C wavelength spectrum range of 100 nm to 280 nm. In some embodiments, the UV light 121 may include the Far UV-C spectrum range of 207 nm to 225 nm.

In the illustrated embodiment, the device 110 includes a button 116 disposed on the device body 111 and in some embodiments, the logic may be configured to activate the UV light source 120 in response to pressing the button 116.

In some embodiments, the device 110 may include a proximity sensor 117 configured to determine a distance between the projection surface 123 and the skin surface 50. The logic may govern activation and/or deactivation of the UV light source 120 based the distance between the projection surface 123 and the skin surface 50 as determined by the proximity sensor 117. For example, the logic may prevent activation the UV light source 120 unless the projection surface 123 is positioned within a predefined distance from the skin surface 50. Similarly, the logic may automatically deactivate the UV light source 120 when the projection surface 123 is moved away from the skin surface 50 such that the distance between the projection surface 123 and the skin surface 50 is greater than the predefined distance. In some embodiments, the logic may deactivate the UV light source 120 after a predefined duration of UV light activation so as to prevent over exposure of the UV light 121.

The device 110 may be a stand-alone unit or the device 110 may be combined with another medical device or apparatus, such as an ultrasound probe, a remote controller of another medical device/system, or an arm band configured to attach/adhere the device 110 to a clinician, for example

The device 110 may include a UV shield 130 coupled with or incorporated into the device body 111, according some embodiments. The UV shield 130 may include a cylindrical wall 132 extending around and away from the projection surface 123 of the device body 111. The UV shield 130 is generally configured to prevent exposure of the UV light 121 to items, people, or portions of the patient 40 other than the insertion site area 51 of the skin surface 50 proximate an open end 131 of the shield 130, i.e., the UV shield 130 is configured to limit UV light exposure to a disinfection area of the skin surface 50 proximate the open end 131 of the UV shield 130. A material of the shield 130 may include one or more of cerium oxide, titanium oxide, carbon black, plastic acrylics, polystyrene, or polycarbonate. As may be appreciated by one of ordinary skill, the UV shield 130 may take any suitable tubular shape, such as cylindrical, polygonal, or oval, for example.

The UV shield 130 may be configured to contact the skin surface 50 at the open end 131. In the illustrated embodiment, a length 133 of the UV shield may define a minimum distance between the skin surface 50 and the projection surface 123, where the minimum distance may limit the intensity of the UV light 121 projected onto the skin surface 50. In some embodiments, the device 110 further may include a contact sensor 118 coupled with the console 115, where the contact sensor 118 is configured to detect a physical contact between the open end 131 of the UV shield 130 and the skin surface 50. In such embodiments, the logic may prevent activation of the UV light source 120 in the absence of the physical contact. Similarly, in some embodiments, the logic may activate the UV light source 120 in response to the physical contact, i.e. when the contact sensor 118 detects the physical contact.

A method of disinfecting a skin surface of a patient may include all or any subset of the following steps, actions or processes, according to some embodiments. The method may include positioning a handheld disinfection device proximate the skin surface, where the hand held device includes a device body and a number of ultraviolet (UV) light sources coupled with the device body, and where the UV light source is configured to project a UV light away from a projection surface of the device body. The handheld disinfection device further includes a console operatively coupled with the UV light source, where the console includes a processor and a memory having stored thereon logic that, when executed by the processor, causes operations of the handheld disinfection device. The method may further include activating the UV light source to disinfect the skin surface.

In some embodiments of the method, activating the UV light source to disinfect the skin surface may be performed in preparation of performing a vascular access procedure, and in some embodiments of the method, activating the UV light source may include pressing a button disposed on the device body.

In some embodiments of the method, the handheld disinfection device further includes a proximity sensor configured to determine a distance between the projection surface and the skin surface, and logic of the handheld disinfection device prevents activation of the UV light source unless the projection surface is positioned within a predefined distance from the skin surface. In such embodiments, the method may further include positioning the projection surface within the predefined distance to allow activation of the UV light source.

In some embodiments of the method, the logic of the handheld disinfection device automatically deactivates the UV light source after a predefined duration of UV light activation.

In some embodiments of the method, the handheld disinfection device includes a UV shield having a cylindrical wall extending around and away from the projection surface of the device body, where the UV shield is configured to limit UV exposure to a disinfection area of the skin surface proximate an open end of the UV shield. The UV shield is configured to contact the skin surface at the open end, and a length of the UV shield to defines a minimum distance between the skin surface and the projection surface.

In some embodiments of the method, the handheld disinfection device further includes a contact sensor coupled with the console, where the contact sensor is configured to detect a physical contact between the open end of the shield and the skin surface. In such embodiments of the method, activating the UV light source includes contacting the skin surface with the open end of the shield.

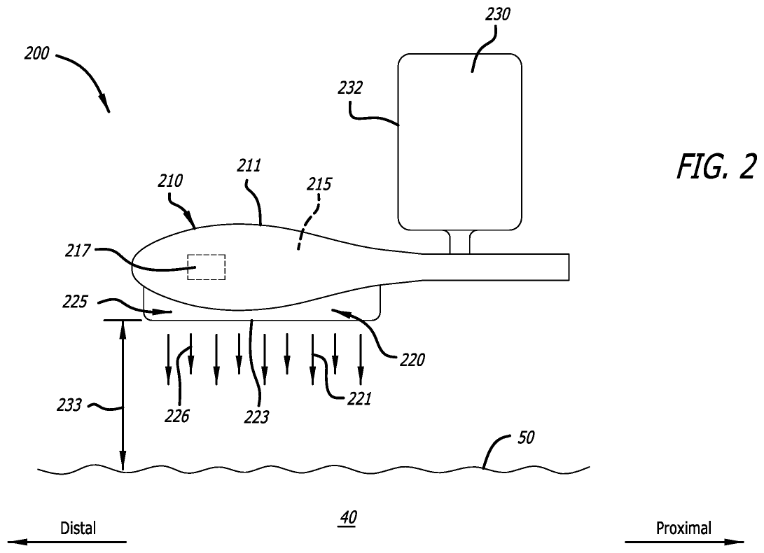

FIG. 2 illustrates a second embodiment of an ultraviolet light disinfection system that can, in certain respects, resemble components of the ultraviolet light disinfection system described in connection with FIG. 1. It will be appreciated that all the illustrated embodiments may have analogous features. Accordingly, like features are designated with like reference numerals, with the leading digits incremented to “2.” For instance, the light source is designated as “120” in FIG. 1, and an analogous light source is designated as “220” in FIG. 2. Relevant disclosure set forth above regarding similarly identified features thus may not be repeated hereafter. Moreover, specific features of the ultraviolet light disinfection system and related components shown in FIG. 1 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the ultraviolet light disinfection system of FIG. 2. Any suitable combination of the features, and variations of the same, described with respect to the ultraviolet light disinfection system and components illustrated in FIG. 1 can be employed with the ultraviolet light disinfection system and components of FIG. 2, and vice versa. This pattern of disclosure applies equally to further embodiments depicted in subsequent figures and described hereafter.

Referring to FIG. 2, the medical disinfection system 200, according to some embodiments, includes a handheld disinfection device 210 having a device body 211 and an ultraviolet (UV) light source 220 coupled with the device body 211. The UV light source 220 is configured to project a UV light 221 away from a projection surface 223 of the device body 211. The device 210 further includes a UV shield 230 disposed proximal the UV light source 220, where the UV shield 230 is configured to prevent passage of the UV light 221 therethrough and allow passage of a visible light (i.e., allow visibility) therethrough. The UV light 220 may be configured to disinfect a skin surface 50 of a patient 40.

The UV shield 230 is positioned between a clinician and the UV light source 220 so that the clinician is protected from exposure to the UV light 221. In some embodiments, the UV shield 230 extends laterally upward away from the device body 221. In some embodiments, a wall 232 of the UV shield 230 is formed of a refractory material such that the UV light 221 entering the wall 232 is prevented from exiting the wall 232.

In some embodiments, the device 210 further includes a visible light source 225 coupled with the device body 211, where the visible light source 225 is configured to project a visible light 226 onto the skin surface 50 to visibly illuminate the skin surface 50. In some embodiments, the visible light source 225 may project the visible light 226 away from the projection surface 223. The visible light source 225 may include one or more of a laser, an optical fiber, a light emitting diode, or any other suitable device configured to emit visible light.

In some embodiments, the device 210 further includes a console 215 operatively coupled with the UV light source 220, where the console 215 includes a processor and a memory having stored thereon logic that governs the operation of the device 210. For example, the logic may deactivate the UV light source 220 after a defined duration. The defined duration may include one of a number of time increments (e.g., 1, 2, 5, or more seconds) stored in the memory or a time increment set by the clinician.

In some embodiments, the device 210 may further include a proximity sensor 217 configured to determine a distance 233 between the projection surface 223 and the skin surface 50. In some embodiments, the logic may prevent activation the UV light source 220 unless the projection surface 223 is positioned within a predefined distance 233 from the skin surface 50.

A method of disinfecting a skin surface of a patient may include all or any subset of the following steps, actions or processes, according to some embodiments. The method may include positioning a handheld disinfection device proximate the skin surface. The hand held device includes a device body and an ultraviolet (UV) light source coupled with the device body, and the UV light source is configured to project a UV light away from a projection surface of the device body. The method may further include (i) activating the UV light source to disinfect the skin surface and (ii) viewing the skin surface through a UV light shield of the handheld disinfection device.

In some embodiments, the method may further include activating a visible light source of the handheld disinfection device to visibly illuminate the skin surface.

In some embodiments of the method, the handheld disinfection device further includes a proximity sensor configured to determine a distance between the projection surface and the skin surface and a console operatively coupled with the UV light source and the proximity sensor. The console includes a processor and a memory having stored thereon logic that, when executed by the processor, causes operations of the handheld disinfection device that include preventing activation of the UV light source unless the projection surface is positioned within a predefined distance from the skin surface. In such embodiments, the method may further include positioning the projection surface within the predefined distance to allow activation of the UV light source.

In some embodiments of the method, the logic of the handheld disinfection device automatically deactivates the UV light source after a defined duration of UV light activation. In some embodiments, the defined duration includes at least one of (i) any one of a number of preset time increments stored in the memory or (ii) a time increment set by the clinician.

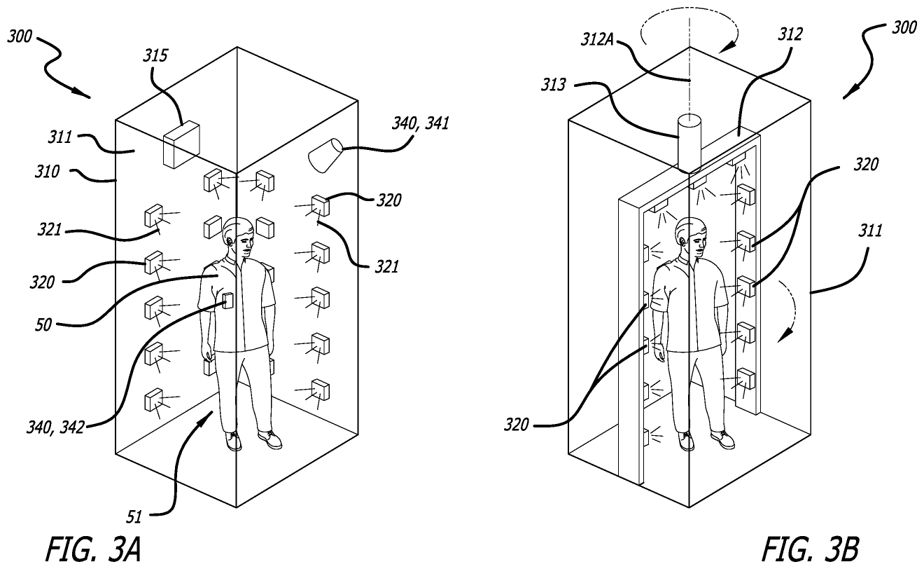

FIG. 3A illustrates a medical disinfection system (system) 300, according to some embodiments. The system 300 is generally configured for disinfecting an exterior of a person such as a medical technician prior to the person entering a controlled medical environment, e.g., an operating room. The system 300 includes an enclosure 310 configured for placement of a person 50 therein and a number (e.g., 1, 2, 5, 10 or more) of ultraviolet (UV) light sources 320 coupled with the enclosure 310, where the UV light sources 320 are configured to project a UV light 321 onto a person exterior 51 of the person 50 to disinfect the person exterior 51. The enclosure 310 includes a perimeter wall 311 defining the enclosure 310. The enclosure 310 may include any suitable shape configured to enclose the person 50 therein. For example, the enclosure 310 may include a rectangular shape having a six sides, i.e. four vertical sides, a top, and a bottom.

In some embodiments, the perimeter wall 311 may be configured to contain the UV light 321 within the enclosure 310, i.e., the perimeter wall 311 may be prevent exposure of the UV light 321 to objects disposed outside the enclosure 310. In some, embodiments the perimeter wall 311 may be formed of a material transparent to visible light. The enclosure 310 may include an opening (not shown), such as a doorway, to enable ingress and egress of the enclosure 310. In some embodiments, the enclosure 310 may include a temporary structure configured for temporary setup and use within a medical environment, where the medical environment may include any environment where a medical procedure may take place, such as a hospital, a clinic, or an emergency medical environment, (.e., a military medical tent), for example.

The UV light sources 320 may be dispersed across an interior of the enclosure 310 in any suitable fashion such that the UV light 321 is projected radially inward onto the person exterior 51. In some embodiments, the UV light sources 320 may be dispersed across substantial entirety of a perimeter wall 311, such that the person 50 is bathed in the UV light 321 (i.e., a substantial entirety of the person exterior 51 is exposed to the UV light 321) when the person 50 is standing within the enclosure 310. In other embodiments, the UV light sources 320 may be coupled with the perimeter wall 311 such that the UV light 321 is projected in one or more discreet directions onto the person exterior 51. In such embodiments, the person 50 may rotate (i.e., turn around) to enable the UV light 321 to be projected onto a substantial entirety of the person exterior 51. By way of one example, the UV light sources 320 may be arranged in a vertical linear array along opposing vertical sides of the perimeter wall 311. In such an embodiment, the person 50 may rotate 180 degrees to enable the UV light 321 to be projected on the substantial entirety of the person exterior 51.

The system 300 may include a console 315 including a processor and a memory (a non-transitory computer-readable medium) having logic stored thereon, where the logic governs operation of the system 300. The console 315 is coupled with the UV lights sources 320 so that the logic may activate and/or deactivate the UV lights sources 320. The logic may be configured to record a disinfecting event, where parameters of the disinfecting event may include one or more of a date of the disinfecting event, a time of the disinfecting event, or activation duration of the UV lights sources 320.

In some embodiments, the system 300 may further include a UV light detection system 340 configured to detect an exposure of the UV light 321 on the person exterior 50, e.g., determine when the person exterior 50 is exposed to the UV light 321. In some embodiments, the UV light detection system 340 may be configured to determine an intensity of the UV light exposure, where the intensity of the UV light exposure may be related to a level of disinfection.

In some embodiments, the UV light detection system 340 may include a first UV light detector 341 (or multiple first UV light detectors 341) physically coupled with the enclosure 310. The first UV light detector 341 may be configured to detect UV light emanating from the person exterior 51, e.g., reflecting off the person exterior 51, and in some embodiments, the first UV light detector 341 may be configured to determine the intensity of the UV light emanating from the person exterior 51, where the UV light emanating from the person exterior 51 may be related to the UV light 321 projected onto the person exterior 51. In some embodiments, the first UV light detector 341 may be configured to scan the person exterior 51 to determine if the substantial entirety of the person exterior 51 is exposed to the UV light 321. For example, the first UV light detector 341 may detect if any portion of the person exterior 51 is not exposed to the UV light 321. In some embodiments, the first light detector 341 may be communicatively coupled with the console 315.

In some embodiments, the UV light detection system 340 may include, in lieu of or in addition to the first UV light detector 341, a second UV light detector 342, where the second light detector 342 is configured to be attached to the person exterior 51. The second UV light detector 342 is configured to detect the UV light 321 received by the person exterior 51. In some embodiments, the UV light detection system 340 may include multiple second UV light detectors 342. In some embodiments, the second light detector 342 may be communicatively coupled with the console 315. The logic may be configured to determine a profile of UV light exposure on the person exterior 51. The profile of UV light exposure may include a coverage assessment of the UV light exposure. In other words, the logic receive signals or data from the first and/or second UV light detectors 341, 342 and determine therefrom what portions of the person exterior 51 have been exposed to the UV light 321 and/or what portions of the person exterior 51 have not been exposed to the UV light 321.

FIG. 3B illustrates a second embodiment of the system 300 further including frame 312 disposed within the enclosure 310. In the illustrated embodiment, the frame 312 defines an inverted U-shape sized to receive the person 50 therein. The UV light sources 320 are attached to the frame 312 such that the UV light sources 320 project the UV light 321 radially inward so as to expose the person exterior 51 to the UV light 321 when the person 50 is positioned within the frame 312. In some embodiments, the UV light sources 320 may be attached to the frame 312 so that the UV light sources 320 form a linear array along the frame 312 thereby defining a plane of the UV light 321. The frame 312 is coupled with the enclosure 310 such that the frame 312 is rotatable about a vertical axis 312A. In some embodiments, the vertical axis 312A may be centrally located within the enclosure 310. As such, during operation, the frame 312 rotates about the person 50 so that the UV light sources 320 travel along a circular path around the person 50, thereby projecting the UV light 321 onto a 360 degree circumference of the person exterior 51. A rotation actuator 313 may be coupled between the frame 312 and the enclosure 310, and the rotation actuator 313 may be operatively coupled with the console 315 so that the logic may govern the operation of the rotation actuator 313.

A method of disinfecting a person exterior may include all or any subset of the following steps, actions or processes, according to some embodiments. The method may include placing the person within an enclosure of a medical disinfection system having a plurality of ultraviolet (UV) light sources coupled with the enclosure, where the UV light sources are configured to project UV light onto a person exterior of the person to disinfect the person exterior; and (ii) activating the UV light sources.

In some embodiments of the method of disinfecting a person, the UV light sources are coupled with a rotatable frame within the enclosure, and the method may further include rotating the frame around the person.

In some embodiments of the method of disinfecting a person exterior, the medical disinfection system further includes a UV light detection system configured to detect a UV light exposure on the person exterior, and the method may further include determining a profile of UV light exposure on the person exterior.

In some embodiments the method of disinfecting a person exterior further includes recording a disinfecting event, where parameters of the disinfecting event include one or more of (i) a date of the disinfecting event; (ii) a time of the disinfecting event; (iii) a duration of the UV light source activation; (iv) an intensity of UV light exposure on the person exterior; or (v) the profile of UV light exposure.

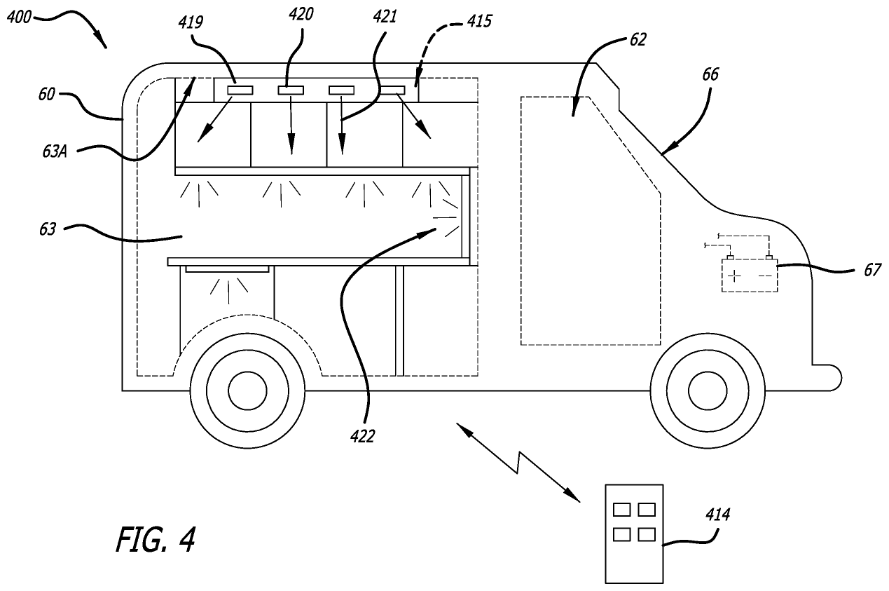

FIG. 4 illustrates a medical disinfection system (system) 400, according to some embodiments. The system 400 is generally configured for disinfecting portions of a patient transport vehicle 60, including objects disposed within the patient transport vehicle 60. The system 400 includes a UV light system 419 coupled with the patient transport vehicle, where the UV light system 419 includes a number (e.g., 1, 2, 5, 10, 20 or more) of ultraviolet (UV) light sources 420 configured to project UV light 421 onto portions of and/or objects disposed within the patient transport vehicle 60, where exposing the objects to the UV light 421 disinfects the objects. The objects may include any surfaces within the patient transport vehicle 60, such as seats, handles, or medical equipment, for example. Further exemplary objects may include tools, instruments, or other medical materials such as drapes, gloves, gowns or other materials for which lack of disinfection may pose a risk to the patient. The patient transport vehicle 60 may include any suitable vehicle for transporting a patient, such as an ambulance or a helicopter, for example. As the UV light sources 420 project and/or disperse the UV light 421 within the patient transport vehicle 60, the patient transport vehicle 60 may be configured to contain the UV light 421 within the patient transport vehicle 60. As such, windows 66 of the patient transport vehicle 60 may be configured to inhibit transmission of the UV light therethrough. Or more specifically, the windows 66 may be configured to prevent transmission the UV light 421 through the windows 66 from the inside to the outside of the windows 66.

The UV light sources 420 may be configured to attach to inside surfaces of the patient transport vehicle 60, and the UV light sources 420 may be configured to project the UV light 421 inward away from the inside surfaces to expose the objects to the UV light 421. In some embodiments, the UV light system 419 may include one or more reflective surfaces 422 configured to reflect the UV light 421 and thereby enhance the exposure of the objects to the UV light 421. As the patient and other medical personal may occupy the patient transport vehicle 60, the UV light system 419 may be configured to activate the UV light sources during periods of non-operation of the patient transport vehicle 60. For example, the system 400 may be configured disinfect the patient transport vehicle 60 between patient transport events.

The UV light system 419 may be configured for selective placement within different arears or compartments of the patient transport vehicle 60, i.e., the UV light system 419 may be portable. Similarly, the UV light system 419 may be configured for selective placement within different patient transport vehicles 60 as needed. In the illustrated embodiment, the UV light system 419 may be portable between at least a first compartment and a second compartment of the patient transport vehicle 60. In some embodiments, the first compartment may include a medical personnel compartment 62 (i.e., a cab of the vehicle) and the UV light system 419 is configured to disinfect objects within the medical personnel compartment 66. In some embodiments, the UV light system 419 may be configured to deactivate the UV light sources 420 when an occupant is present within the medical personnel compartment 66 to prevent exposure of the occupant to the UV light 421. In some embodiments, the second compartment may include a patient compartment 63 and the UV light system 419 may be configured to disinfect objects within the patient compartment 63. In some embodiments, the UV light sources 420 may be coupled with an overhead ceiling surface 63A of the patient compartment 63.

In some embodiments, the UV light system 419 may include a remote control 414 configured to enable a user of the system 400 to remotely activate and deactivate the UV light sources 420. In some embodiments, the UV light system 419 may include a timer 415 operatively coupled with the UV light sources 420, where the timer 415 may be configured to at least deactivate the UV light sources 420 after a defined activation time period. In some embodiments, the timer 415 may also be configured to activate the UV light sources 420 according to a defined schedule or time increments. The UV light system 419 may be electrically coupled with an electrical system 67 of the patient transport vehicle 60. In some embodiments, the UV light system 419 may include a battery (not shown) in leu of or in addition to being coupled with the electrical system 67.

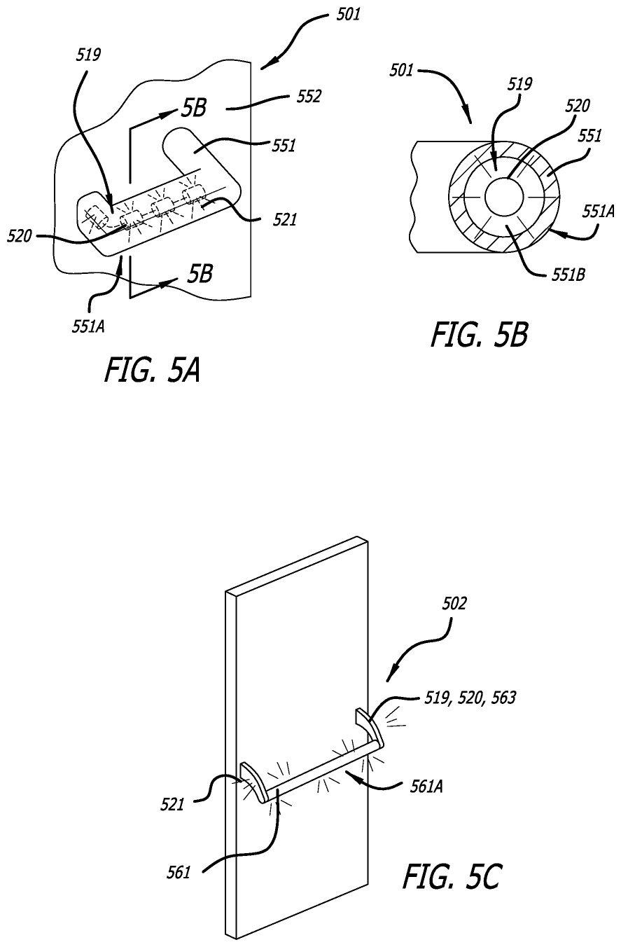

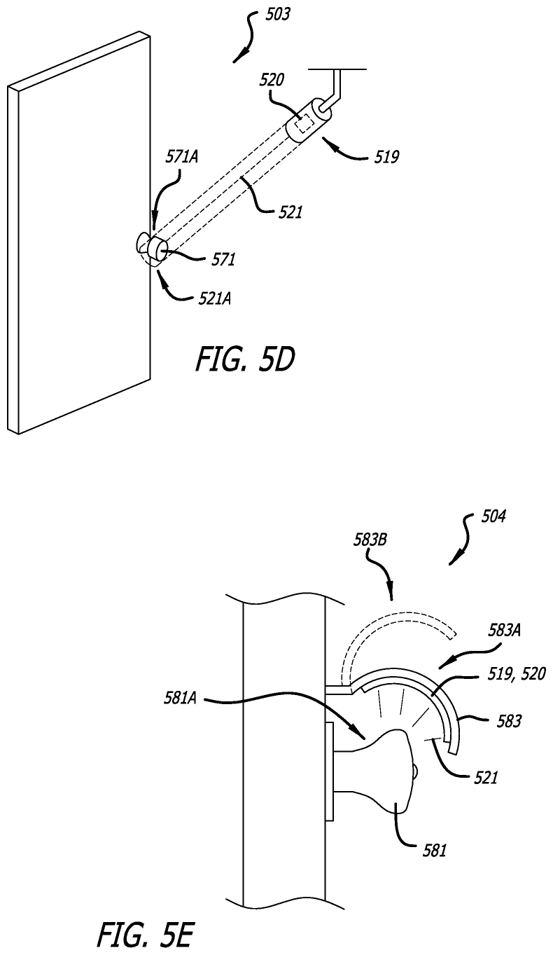

FIGS. 5A-5E illustrate various embodiments of medical disinfection systems configured to disinfect various high-touch surfaces of a patient care facility. The high-touch surfaces may include any surface within the patient care facility. In some implementations, the high-touch surfaces may be located within a patient care environment, such as an operating room, for example. In other implementations, the high-touch surfaces may be located within a sterile controlled environment, such as an intravenous preparation environment of a pharmacy, for example. The various medical disinfection systems are configured to disinfect the high-touch surfaces by exposing the high-touch surfaces to an ultraviolet light.

FIG. 5A illustrates a perspective view of a medical disinfection system 501 configured to expose a high-touch surface 551A of a handle 551 of a door 552. FIG. 5B is a cross sectional view of the handle 551 of FIG. 5A cut along the line sectioning lines 5B-5B. With reference to FIGS. 5A and 5B, the medical disinfection system 501 includes a UV light system 519 configured to project a UV light 521 outward from an interior of the handle 551 through a transparent material of the handle 551. More specifically, the high-touch surface 551A is an exterior surface of the handle 551, where the handle 551 includes (or is formed of) a material that is transparent to the UV light 521. The UV light system 519 includes a number (e.g., 1, 2, 3, or more) of UV light sources 520 that are disposed (e.g., within a cavity 551B) beneath the exterior surface of the handle 561 such that the UV light 521 propagates through the transparent material to the high-touch surface 551A. As such, the UV light sources 520 are optically coupled with the high-touch surface 551A. In some embodiments, the handle 551 may include a latch.

FIG. 5C illustrates a perspective view of a medical disinfection system 502 configured to expose a high-touch surface 561A of a handle 561. In the illustrated embodiment, the handle 561 defines an elongate shape defining a length of the handle 561. Similar to the medical disinfection system 501, the medical disinfection system 502 includes the UV light system 519 configured to project a UV light 521 outward from an interior of the handle 561 through a transparent material of the handle 561. The UV light system 519 includes a number (e.g., 1, 2, 3, or more) of UV light sources 520 disposed within a cavity 561B of the handle 561. The UV light sources 520 may further include an optical fiber 563 or number (e.g., 1, 2, 3, or more) of optical fibers 563. In some embodiments, the optical fiber 563 is configured to project the UV light 521 laterally (e.g., radially) away from the optical fiber 563 along a length of the optical fiber 563. The optical fiber 563 extends along the length of the handle 561 so that the optical fiber 563 projects the UV light 521 radially outward from the optical fiber 563 along the length of the handle 561, thereby disinfecting the high-touch surface 561A along a substantial entirety of the length of the handle 561.

FIG. 5D illustrates a perspective view of a medical disinfection system 503 configured to expose a high-touch surface 571A of a handle 571 (e.g., a door knob as illustrated). The UV light system 519 includes a UV light source 520 or a number (e.g., 1, 2, 3, or more) of UV light sources 520 disposed away from the handle 571. The UV light source 521 is configured to project the UV light 521 onto the high-touch surface 571A from a distance away from the high-touch surface 571A. For example, the UV light source 520 may be attached to a ceiling surface. The UV light source 520 is configured focus the UV light 521 onto the high-touch surface 571A so that only an area 521A adjacent the high-touch surface 571A and a surface adjacent the high-touch surface 571A are exposed to the UV light 521. By focusing the UV light 521, objects or materials that may be damaged by exposure to the UV light 521 are prevented from exposure to the UV light 521.

FIG. 5E illustrates a side view of a medical disinfection system 503 configured to expose a high-touch surface 581A of a handle 581 (e.g., a door knob as illustrated). The UV light system 519 includes a UV light source 520 or a number (e.g., 1, 2, 3, or more) of UV light sources 520 configured to expose the high-touch surface 581A to the UV light 521. The medical disinfection system 504 includes a shroud 583 configured to at least partially cover or encapsulate the high-touch surface 581A. The UV light source 521 is attached to an underside (or inside) surface of the shroud 583 and the UV light source 521 is configured to project the UV light 521 away from the inside surface of the shroud 583 to the high-touch surface 581A. In some embodiments, the inside surface includes a reflective surface configured to enhance the exposure of the high-touch surface 581A to the UV light 521.

In some embodiments, the shroud 583 is configured to transition between a deployed position 583A and a retracted position 583B (shown in phantom lines). In the deployed position 583A, the shroud 583 may substantially cover (or encapsulate) the high-touch surface 581A so that the UV light source 521 may effectively expose a substantial entirety of the high-touch surface 581A to the UV light 521. Conversely, in the retracted position 583B, the shroud 583 may substantially uncover the high-touch surface 581A, i.e., the shroud 583 may be disposed sufficiently away from the high-touch surface 581A, to allow the user to access to the handle 581.

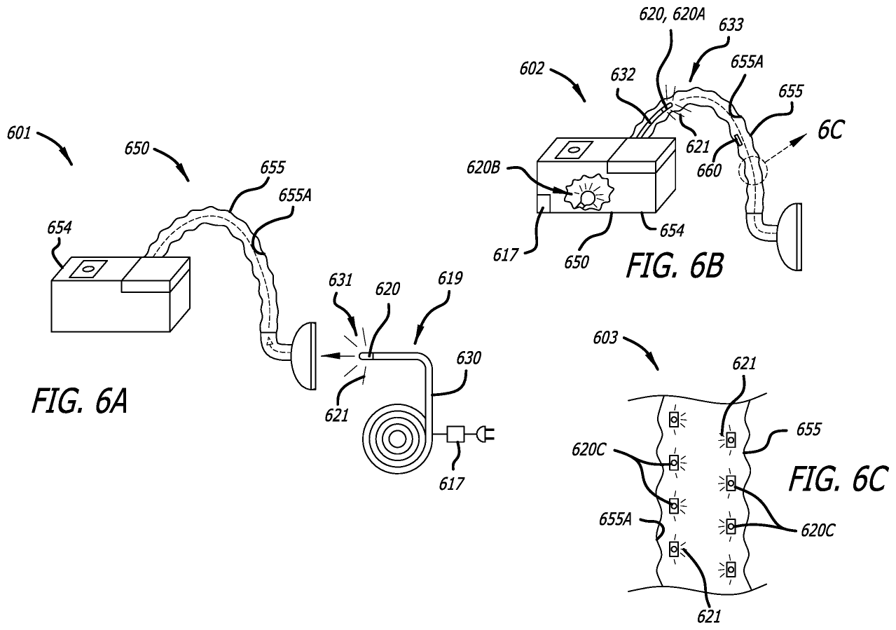

FIGS. 6A-6C illustrate various embodiments a medical disinfection system configured to disinfect a continuous positive airway pressure (CPAP) system 650. More specifically, the medical disinfection system is configured to disinfect an interior 655A (i.e., the inside surfaces) of a tube 655 of the CPAP system 650. According to some embodiments, the medical disinfection system includes an ultraviolet (UV) light system 619 configured to disinfect the interior 655A of the tube 655 by exposing the interior 655A to the UV light 621. A material of the tube 655 may be compatible with exposure to the UV light 621. In other words, the tube 655 may generally be configured to withstand UV light exposure without damage or degradation.

FIG. 6A illustrates a medical disinfection system 601 that includes the CPAP system 650 including a CPAP module 654 operatively coupled with the tube 655. The UV light system 619 is coupled with a CPAP system 650 during use of the UV light system 619. The UV light system 619 includes an elongate member 630 having a UV light source 620 or a number (1, 2, 3, or more) of UV light sources 620 coupled therewith. During use, the elongate member 630 including the UV light source 620 is inserted into the tube 655 so that the UV light source 620 can project the UV light 621 onto the interior 655A, i.e., expose the interior 655A to the UV light 621. In some embodiments, the UV light source 620 may be located at a free end 631 of the elongate member 630. The elongate member 630 may include a stylet, a probe, a scope, or any other suitable elongate device configured for placement within the tube 655. The UV light system 619 may receive electrical power from a facility power source or the UV light system 619 may be battery operated, i.e. include a battery (not shown).

The UV light source 620, including the elongate member 630, is configured to be selectively inserted within the tube 655 and removed from the tube 655. In some embodiments, the user may insert the UV light source 620 into the tube 655 to disinfect the tube 655 between CPAP events. In some embodiments, the UV light source 620 includes a single light source 620 at the free end 631. In such embodiments, the UV light source 620 may disinfect portions of segments of the interior 655A during advancement or retraction of the UV light source 620 along the length of the tube 655. In other embodiments, the UV light source 620 includes a plurality of UV light sources 620 disposed along a length of the elongate member 630. In such other embodiments, the UV light source 620 may disinfect the interior 655A after placement of the elongate member 630 within the tube 655, i.e., while the elongate member 630 resides within the tube 655.

In some embodiments, the UV light system 619 may include a notification system 617 configured to indicate a status of a disinfection cycle of the UV light system 619. In some embodiments, the notification system 617 may include a light or other visual indicator configured to indicate the status of the disinfection cycle (e.g., an “on” of “off” condition of the UV light source 620). In other embodiments, the notification system 617 may include an audible indicator to indicate the status of the disinfection cycle. In still other embodiments, the notification system 617 may include both the visual indicator and the audible indicator.

FIG. 6B illustrates a medical disinfection system 602, according to other embodiments, that may resemble some features and functionalities of the medical disinfection system 601 of FIG. 6A. The medical disinfection system 602 includes the CPAP system 650 including the CPAP module 654 operatively coupled with the tube 655. The UV light system 619 is incorporated into the CPAP system 650 such that the CPAP module 654 governs operation of the UV light system 619. The UV light system 619 includes an elongate member 632 having a UV light source 620 or a number (1, 2, 3, or more) of UV light sources 620 coupled therewith. During use, the elongate member 630 including the UV light source 620 is advanced along the tube 655 so that the UV light source 620 can project the UV light onto the interior 655A, i.e., expose the interior 655A to the UV light 621. In some embodiments, the UV light source 620 may be located at a free end 633 of the elongate member 632. The elongate member 632 may include a stylet, a probe, a scope, or any other suitable elongate device configured for advancement within the tube 655. The UV light system 619 receives electrical power from CPAP module 654.

The UV light source 620, including the elongate member 632, is configured for automatic advancement within the tube 655 and retraction from the tube 655. More specifically, an actuator (not shown) of CPAP module 654 may insert the UV light source 620 into the tube 655 to disinfect the tube 655 between CPAP events. In some embodiments, the UV light source 620 includes a single light source 620 at the free end 631, such that the UV light source 620 disinfects portions or segments of the interior 655A during advancement and/or retraction of the UV light source 620 along the length of the tube 655. In other embodiments, the UV light source 620 includes a plurality of UV light sources 620 disposed along a length of the elongate member 632. In such other embodiments, the UV light source 620 may disinfect the interior 655A after advancement of the elongate member 632 within the tube 655 and before retraction of the elongate member 632 from the tube 655. In some embodiments, the CPAP module 654 may contain the elongate member 632 when the elongate member 632 is not disposed within the tube 355, i.e., between disinfecting events.

In some embodiments, the light source 620 may include a light emitting diode (LED) 620A at the free end 633. In other embodiments, the light source 620 may include a light emitting diode 620B disposed within the CPAP module 654. In such other embodiments, the elongate member 632 may be configured to propagate the UV light 621 along the elongate member 632 and project the UV light 621 away from the elongate member 632. In some embodiments, the light source 620 may include an optical fiber extending along the elongate member 632. In some embodiments, the notification system 617 may be incorporated into the CPAP system 650.

As the UV light system 619 is incorporated into the CPAP system 650, the UV light system 619 may be configured to automatically initiate the disinfection cycle in accordance with operation of the CPAP system 650. For example, UV light system 619 may automatically initiate a disinfection cycle between CPAP events or according to a defined schedule, such as during periods of non-sleep of the user.

In some embodiments, the UV light system 619 may include a sensor 660 configured to detect a contamination of the tube 655. In such embodiments, the UV light system 619 may be configured to initiate the disinfection cycle based on a detection of the contamination. For example, the UV light system 619 may be configured to initiate the disinfection cycle only if or when the contamination of the tube 655 is detected. The sensor 660 may include any suitable sensor for detecting the contamination, such as an optical sensor, a capacitance sensor, an inductance sensor, or an ultrasonic sensor, for example.

FIG. 6C illustrates a medical disinfection system 603, according to other embodiments, that may resemble some features and functionalities of the medical disinfection system 602 of FIG. 6B. FIG. 6C shows a detailed cross-sectional view of a portion of the tube 655 of FIG. 6B. In leu of or in addition to the elongate member 632, the UV light system 619 may include a plurality of UV light sources 620C disposed along the interior 655A of the tube 655. In such embodiments, the UV light sources 620C may be fixedly attached to the interior 655A (i.e., the inside surface) of the tube 655. The UV light sources 620C may be configured to expose an entirety of the interior 655A to the UV light 621 to disinfect the entirety of the interior 655A. The UV light sources 620C may be electrically coupled with and receive power from the CPAP module 654.

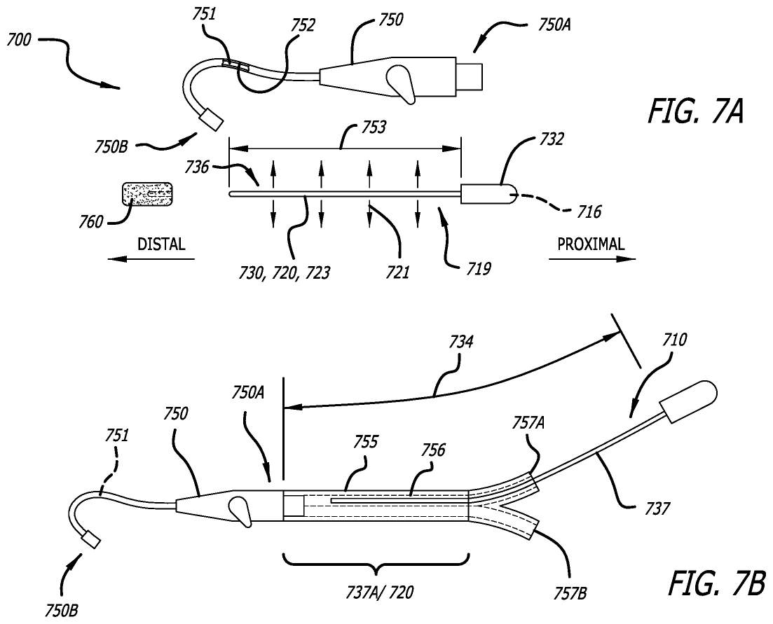

FIG. 7A illustrates an exploded view of a medical disinfection system configured to disinfect a saliva ejector, according to some embodiments. The medical disinfection system 700 includes an ultraviolet (UV) light system 719 coupled with a saliva ejector 750 defining a lumen 751 having a lumen length extending between a proximal end 750A and a distal end 750B of the saliva ejector 750. The lumen 751 defines an inside surface 752. The UV light system 719 includes an elongate member 730 configured for placement within the lumen 751. The elongate member 730 may include a handle 732 to enable a user to manipulate the elongate member 730. A length 733 of the elongate member may be equal to or greater than the length of the lumen 751. During use, the elongate member 730 may be inserted into the lumen 751 from the proximal end 750A or the distal end 750B.

The UV light system 719 includes a UV light source 720 coupled with or incorporated into the elongate member 730. In the illustrated embodiment, the UV light source 720 includes an optical fiber 723 which may extend along the length 733. The UV light source 720 is generally configured to expose the inside surface 752 to a UV light 721, where the UV light 721 is configured to disinfect the inside surface 752. The optical fiber 723 is configured to project the UV light 721 radially outward from the optical fiber 723 along the length 733. In some embodiments, the optical fiber 723 includes a coating 736 configured to disperser the UV light 721 within the lumen 751. In some embodiments, the UV light system 719 may be configured for single use such that the UV light system 719 is discarded after the single use. In other embodiments, the UV light system 719 may be configured for repeated use.

In some embodiments, the medical disinfection system 700 further includes a liquid absorbing device 760. The liquid absorbing device 760 may be attached to the elongate member 730, such as at a distal end of the elongate member 730, for example. The liquid absorbing device 760 is configured to remove a liquid (e.g., residual saliva) from the lumen 751. In other words, the liquid absorbing device 760 is configured to dry the lumen 751. In some embodiments, the liquid absorbing device 760 includes a cloth member configured to swab the inside surface 752 during insertion of the elongate member 730 into the lumen 751 and/or removal of the elongate member 730 from the lumen 751. In some embodiments, the liquid absorbing device 760 may be configured for attachment to and detachment from the elongate member 730 by the user.

FIG. 7B illustrates another embodiment of the medical disinfection system 700 where the saliva ejector 750 further includes a casing 755 extending proximally away from the proximal end 750A. The casing 755 includes a casing lumen 756 in fluid communication with the lumen 751. An elongate member port 757A and a vacuum part 757B are each in fluid communication with the casing lumen 757. The elongate member 737 has a length 734 sufficient to extend between the elongate member port 757A and the distal end 750A. The UV light source 720 extends along at least a distal portion 737A of the elongate member 737.

The UV light system 719 is transitionable between a use state and a storage state. In the storage state, the elongate member 734 is disposed in a proximal position such that the distal portion 737A and the UV light source 720 are disposed within the casing lumen 756 as illustrated in FIG. 7B. In the use state, the elongate member 734 is disposed in a distal position such that the distal portion 737A and the UV light source 720 are disposed within the lumen 751. The UV light system 719 is configured for repeated use, i.e., the UV light source 720 is configured for repeatable placement within and removal from the lumen 751 so as to repeatedly disinfect the inside surface 752.

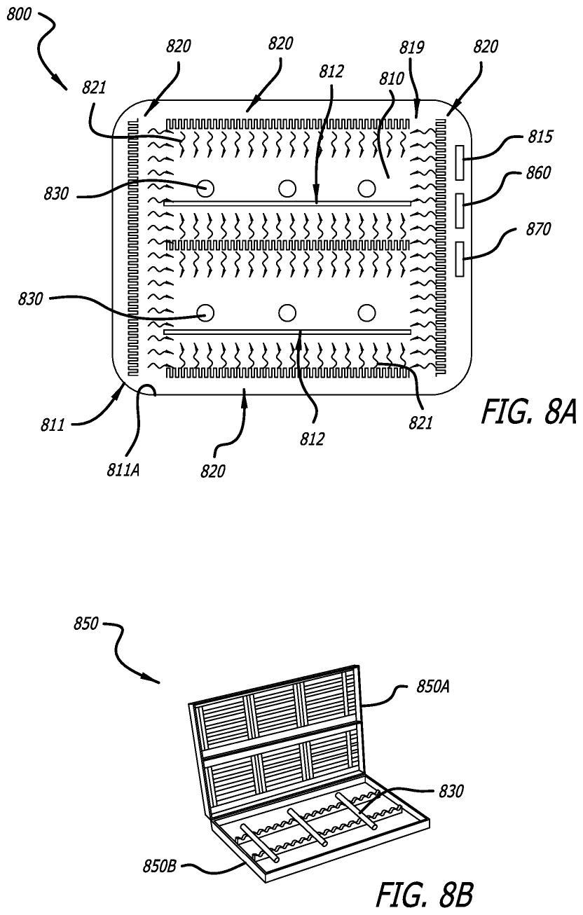

FIG. 8A illustrates a medical disinfection system configured to disinfect medical devices (e.g., dental instruments), according to some embodiments. The medical disinfection system (system) 800 includes an enclosure 810 configured for placement of medical devices 830 therein and an ultraviolet (UV) light source 820 coupled with the enclosure 810. The UV light source 820 is configured to expose the medical devices 830 to a UV light 821 to disinfect the medical devices 830.

The enclosure 810 includes a perimeter wall 811 configured to contain the UV light 821 within the enclosure 810. In some embodiments, the UV light source 820 includes a plurality of UV light sources 820 that are dispersed across an interior surface 811A of the perimeter wall 811. As such, the UV light sources 820 are configured to project the UV light 821 inward onto the medical devices 830.

The enclosure 810 may include a number of shelves 812 disposed within the enclosure 810 and the shelves 812 are configured for placement of the medical devices 830 thereon. In some embodiments, one or more of the UV light sources 820 are disposed between adjacent shelves 812.

In some embodiments, at least a first subset of the UV light sources 820 include light emitting diodes (not shown). In some embodiments, at least a second subset of the plurality of UV light sources 820 includes optical fibers (not shown), where each optical fiber is configured to project the UV light 821 laterally away from the optical fiber so as to expose the medical devices 830 with the UV light 821.

The medical disinfection system 800 may include a console 815 that is operatively coupled with the UV light source 820. The console 815 includes a processor and a memory (e.g., a non-transitory computer-readable medium) having stored thereon logic that, when executed by the processor, causes operations of the medical disinfection system 800. The operations may include at least activating the UV light source 820. In some embodiments, the operations further include receiving input from a user and adjusting a duration of UV light activation based on the input from the user.

In some embodiments, the system 800 further includes a medical device identification system 860. The medical device identification system 860 is configured to identify at least one medical device 830 or more than one medical device 830 disposed within the enclosure 810. The medical device identification system 860 may include any suitable identification mechanism, such as a bar code reader, or a radio frequency identification reader, for example. According to one embodiment, the medical device identification system 860 may include a camera (not shown) coupled with the console 815 where the camera is configured to obtain an image of the at least one medical device 830. The logic may be configured to compare the obtained image with medical device images stored in the memory. Based on the comparison, the medical device identification system 860 may identify the at least one medical device 830. In some embodiments, the logic may automatically adjust the duration of UV light activation based on the identification of the at least one medical device 830.

In some embodiments, the medical disinfection system 800 may further include a temperature control system 870. The temperature control system 870 may including a heating system configured to warm the medical devices 830 disposed within the enclosure 810 and/or a cooling system (e.g., a refrigeration system) configured to cool the medical devices 830 disposed within the enclosure 810.

FIG. 8B illustrates a cassette (or tray) 850 configured for placement within the enclosure 810, where the cassette 850 is also configured for placement of the medical devices 830 therein. The cassette 850 is configured for transmission of the UV light 821 therethrough. In some embodiments, the cassette 850 may be formed of a material that is transparent to the UV light 821. In use, the medical devices 830 are placed within one or more cassettes 850 and the cassettes 850 are placed within the enclosure 810. The cassette 850 may include a base 850A hingedly coupled with a cover 850B.

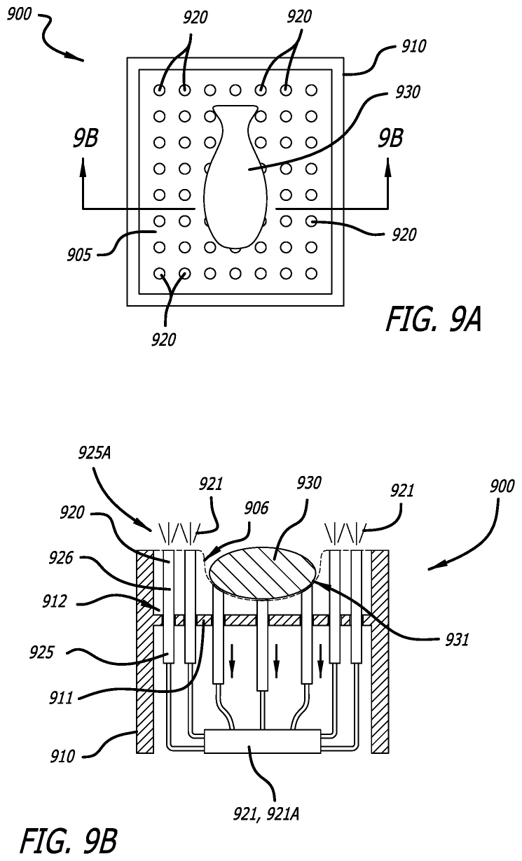

FIGS. 9A and 9B illustrate a medical disinfection system 900. FIG. 9A is a top view illustration of the medical disinfection system 900 in use with a medical device 930, according to some embodiments. FIG. 9B is a cross-sectional side view of the medical disinfection system 900 including the medical device 930 cut along sectioning lines 9B-9B. Referencing FIGS. 9A and 9B, the medical disinfection system 900 includes a plurality of UV light sources 920 arranged in a two-dimensional array and a frame 910 coupled with the plurality of UV light sources 920. The frame 910 defines a disinfection area 905 and the plurality of UV light sources 920 define a deflectable surface 906 (e.g., a bed of UV lights) extending across the disinfection area 905, where deflectable surface 906 is configured for placement of the medical device 930 thereon. The deflectable surface 906 is configured to conform to an external surface 931 of the medical device 930 when the medical device 930 is placed on the deflectable surface 906. The UV light sources 920 are configured to project a UV light 921 away from the deflectable surface 906 onto the external surface 931 to disinfect the external surface 931.

In some embodiments, each UV light source 920 includes an elongate member 925 (e.g., an elongate pin), and each UV light source 920 is configured to project the UV light 921 away from a free end 925A of the elongate member 925. In some embodiments, each UV light source 920 is directly coupled with the frame 910. In some embodiments, the frame 910 includes a plate 911 extending across the disinfection area 905, and each UV light source 920 is individually displaceable with respect to the plate 911. The plate 911 includes a plurality of apertures 912 extending through the plate 911, and each elongate member 925 is slidably disposed within a corresponding aperture 912. In some embodiments, each UV light source 920 includes a light guide 926 (e.g., an optical fiber) extending along the elongate member 925. In some embodiments, the medical disinfection system 900 may include a light module 921 that includes a number of UV light emitting devices 921A (e.g., light emitting diodes) optically coupled with the UV light sources 920.

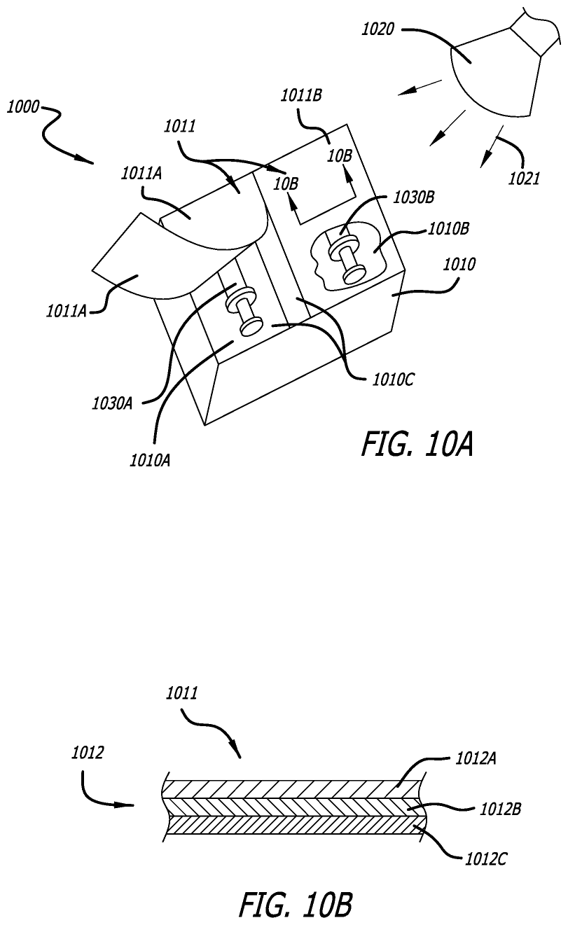

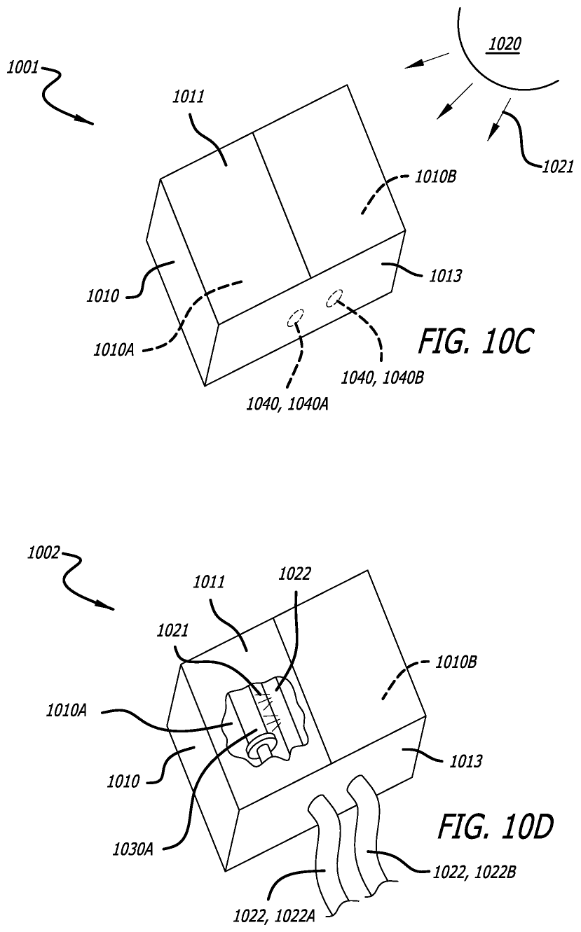

FIG. 10A illustrates a perspective view of a medical device package (package) 1000, according to some embodiments. The package 1000 is generally configured to enable disinfection of one or more medical devices within the package 1000 via exposure to an ultraviolet (UV) light 1021 as may be projected from an external UV light source 1020. As such, the package 1000 is configured for exposure to the UV light 1021 where the UV light 1021 is configured for the disinfection of medical devices.

The package 1000 generally includes a container 1010 and a cover 1011. The container 1010 includes a number (e.g., 1, 2, 3, or more) of compartments such as a first compartment 1010A and a second compartment 1010B. A first medical device 1030A is disposed within the first compartment 1010A and a second medical device 1030B is disposed within the second compartment 101B. Each of the first and second medical devices 1030A, 1030B are configured to be disinfected (e.g., sterilized) via exposure to a dose of the UV light 1021. In some embodiments, a dose of UV light is defined by a combination of UV light intensity and a duration of exposure. In some embodiments, a medical device may require at least a defined dose (or dose range) of UV light in order to be adequately disinfected. Further in some embodiments, the medical device may be damaged by a dose of UV light that exceeds the defined dose (or dose range). Further still, some medical devices may require a greater dose than other medical devices. In the illustrated embodiment, the first medical device 1030A is configured to receive a first dose of the UV light 1021 to disinfect the first medical device 1030A, and the second medical device 1030B is configured to receive a second dose of the UV light 1021 to disinfect the second medical device 1030B. For purpose of description, the second dose is greater than the first dose. The container 1010 is configured to facilitate exposure of the first and second medical devices 1030A, 1030B to the UV light 1021. The inside wall surfaces 1010C may be configured to reflect the UV light 1021 so that a substantial entirety of the first and second medical devices 1030A, 1030B are exposed to the UV light 1021.

The cover 1011 is generally configured to retain the first and second medical devices 1030A, 1030B within the first and second compartments 1010A, 1010B, respectively. The cover 1011 is removeably coupled with the container 1010 so as to extend across open ends (e.g., open top ends) of the first and second compartments 1010A, 1010B. In the illustrated embodiment, the cover 1011 includes a first cover portion 1011A extending across the open end of the first compartment 1010A and a second cover portion 1011B extending across the open end of the second compartment 1010B. In use, a user may peel the cover 1011 from the container 1010 to gain access to the first and second medical devices 1030A, 1030B. The first cover portion 1011A and the second cover portion 1011B may be removed individually from the first and second compartments 1010A, 1010B, respectively.

In some embodiments, the cover 1011 may be configured to allow transmission of the UV light 1021 through the cover 1011. In the illustrated embodiment, the first cover portion 1011A is configured to allow the first dose of the UV light 1021 to enter the first compartment 1010A (i.e., pass through the first cover portion 1011A) to disinfect the first medical device 1030A. Similarly, the second cover portion 1011B is configured to allow the second dose of the UV light 1021 to enter the second compartment 1010B (i.e., pass through the second cover portion 1011A) to disinfect the second medical device 1030B.

FIG. 10B is a cross-section detail view of a portion of the cover 1011 as cut along the sectioning lines 10B-10B of FIG. 10A. In some embodiments, the cover 1011 includes a number (e.g., 1, 2, 3, or more) layers 1012, such as a first layer 1012A, a second layer 1012B, and third layer 1012C, for example. The first layer 1012A, the second layer 1012B, and third layer 1012C are configured to be individually removed from the container 1010. For example, in a first instance, the user may remove the first layer 1012A leaving the second and third layers 1012B, 1012C in place. In a second instance, the user may remove the first and second layers 1012A, 1012B leaving the third layer 1012C in place.

The layers 1012 may each be configured to limit or control the transmission of the UV light 1021 through the cover 1011. For example, the first layer 1012A may be configured to prevent the transmission of the UV light 1021, and the second and third layers 1012B, 1012C may be configured to allow a limited transmission of the UV light 1021. By way of example, the user may remove the first layer 1012A from the first cover portion 1011A. In such an instance, the remaining second and third layers 1012B, 1012C may allow the first dose of UV light 1021 to enter the first compartment 1010A to disinfect the first medical device 1030A. By way of another example, the user may remove the first and second layers 1012A, 1012B from the second cover portion 1011B. In such an instance, the remaining third layer 1012C may allow the second dose of UV light 1021 to enter the second compartment 1010B to disinfect the second medical device 1030B.

FIG. 10C illustrates another embodiment of a medical device package. The medical device package (package) 1001 may include features and functionalities that resemble the package 1000 of FIGS. 10A, 10B. The package 1001 includes, in addition to or in lieu of the layers 1012B, 1012C of FIG. 10B, a number of UV light pathways 1040 that enable the UV light 1021 to enter container 1010. In some embodiments, the UV light pathways 1040 may pass through an external wall 1013 and/or the cover 1011. Each of the first and second compartments 1010A, 1010B may include one or more UV light pathways 1040 such as the first UV light pathway 1040A and the second UV light pathway 1040B. In some embodiments, the UV light pathways 1040 may includes one or more of a tube, a window, a light pipe, or an optical fiber. In some embodiments, a different subsets of UV light pathways 1040 may be configured transmit different doses of the UV light 1021. For example, the first UV light pathway 1040A may be configured to transmit (i.e., allow entry of) the first dose of UV light 1021 into the first compartment 1010A. Similarly, the second UV light pathway 1040B may be configured to transmit the second dose of UV light 1021 into the second compartment 1010A.

FIG. 10D illustrates another embodiment of a medical device package. The medical device package (package) 1002 may include features and functionalities that resemble the packages 1000, 1001 of FIGS. 10A-10C. The package 1002 includes, in addition to or in lieu of the layers 1012B, 1012C of FIG. 10B and/or the UV light pathways 1040 of FIG. 10C, a number of UV light probes 1022 configured to emit the UV light 1021 within the container 1010. In some embodiments, the UV light probes 1022 may pass through an external wall 1013 and/or the cover 1011. Each of the first and second compartments 1010A, 1010B may include one or more UV light probes 1022 such as the first UV light probe 1022A and the second UV light probe 1022B. In some embodiments, the UV light probes 1022 may include one or more of a light emitting diode or an optical fiber. In some embodiments, a different subsets of UV light probes 1022 may be configured emit different doses of the UV light 1021. For example, the first UV light probe 1022A may be configured to emit the first dose of UV light 1021 into the first compartment 1010A. Similarly, the second UV light probe 1022B may be configured to transmit the second dose of UV light 1021 into the second compartment 1010A.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly. departures may be made from the particular embodiments disclosed herein without departing from the scope of the concepts provided herein.

1. A medical disinfection system, comprising:
a handheld disinfection device, comprising:
a device body;
an ultraviolet (UV) light source coupled with the device body, the UV light source configured to project a UV light away from a projection surface of the device body; and
a console operatively coupled with the UV light source, the console including a processor and a memory having stored thereon logic that, when executed by the processor, causes operations, including:
activating the UV light source to project the UV light; and
deactivating the UV light source.

2. The system according to claim 1, wherein the UV light is configured to disinfect a skin surface of a patient prior to breaching a dermal layer.

3. The system according to claim 2, wherein breaching the dermal layer includes accessing a vasculature.

4. The system according to claim 1, wherein the UV light includes a wavelength within the range of 207 nm to 222 nm.

5. The system according to claim 1, wherein the operations include activating the UV light source in response to pressing a button disposed on the device body.

6. The system according to claim 1, wherein:
the handheld disinfection device further includes a proximity sensor configured to determine a distance between the projection surface and the skin surface, and the operations further include preventing activation the UV light source unless the projection surface is positioned within a predefined distance from the skin surface.

7. The system according to claim 1, wherein the operations further include deactivating the UV light source after a predefined duration of UV light activation.

8. The system according to claim 1, wherein the handheld disinfection device is combined with at least one of an ultrasound probe, a remote controller, or an adherable arm band.

9. The system according to claim 1, wherein the handheld disinfection device includes a UV shield having a cylindrical wall extending around and away from the projection surface of the device body, the UV shield configured to limit UV exposure to a disinfection area of the skin surface proximate an open end of the UV shield.

10. The system according to claim 9, wherein:
the UV shield is configured to contact the skin surface at the open end, and
a length of the UV shield to defines a minimum distance between the skin surface and the projection surface.

11. The system according to claim 10, wherein:
the handheld disinfection device further includes a contact sensor coupled with the console, the contact sensor configured to detect a physical contact between the open end of the shield and the skin surface, and
the operations further include at least one of:
preventing activation of the UV light source in the absence of the physical contact, or
activating the UV light source in response to the physical contact.

12. The system according to claim 9, wherein a material of the shield includes one or more of cerium oxide, titanium oxide, carbon black, plastic acrylics, polystyrene, or polycarbonate.

13-19. (canceled)

20. A medical disinfection system, comprising:
a handheld disinfection device, comprising:
a device body;
an ultraviolet (UV) light source coupled with the device body, the UV light source configured to project a UV light away from a projection surface of the device body; and
a UV shield disposed proximal the UV light source, the UV shield configured to:
prevent passage of UV light therethrough, and
allow passage of visible light therethrough.

21. The system according to claim 20, wherein the UV shield is positioned between the clinician and the UV light source so that a clinician is protected from exposure to the UV light.

22. The system according to claim 20, wherein the UV shield extends laterally upward away from the device body.

23. The system according to claim 20, wherein a wall of the UV shield is formed of a refractory material such that UV light entering the wall is prevented from exiting the wall.

24. The system according to claim 20, wherein the UV light is configured to disinfect a skin surface of a patient.

25. The system according to claim 20, wherein the handheld disinfection device further includes a visible light source coupled with the device body, the visible light source configured to visibly illuminate the skin surface.

26. The system according to claim 20, wherein the device further includes a console operatively coupled with the UV light source, the console including a processor and a memory having stored thereon logic that, when executed by the processor, causes operations, including deactivating the UV light source after a defined duration.

27. The system according to claim 26, wherein the defined duration includes at least one of:
any one of a number of preset time increments stored in the memory, or a time increment set by the clinician.

28. The system according to claim 26, wherein:
the handheld disinfection device further includes a proximity sensor configured to determine a distance between the projection surface and the skin surface, and
the operations further include preventing activation the UV light source unless the projection surface is positioned within a predefined distance from the skin surface.

29-129. (canceled).
